SEQUENCE LISTING (16 sequences)

SEQ ID NO: 1 - DNA, 300 bp, Staphylococcus aureus
tatattatgtctcattttcttcaatatgtacttatttatattttaccgtaatttactatatttagttgcagaaagaattttctcaaagctagaactttgcttcactataagtattcagtataaagaatatttcgctattatttacttgaaatgaaagactgcggaggctaactatgtcaaaaatcatgaacctcattacttatgataagcttcttaaaaacataacagcaattcacataaacctcatatgttctgatacattcaaaatccctttatgaagcggctgaaaaaaccgcatca

SEQ ID NO: 2 - DNA, 300 bp, Staphylococcus aureus
aatctaataaacttccttttattaatcgtaggcattgtatatttcctttcattctttcttgattccattagtttaaatttaaaatttcatcatcaatttcttaatttaattgtagttccataatcaatataatttgtacagttattatatattctagatcatcaatagttgaaaaatggtttattaaacactctataaacatcgtatgatattgcaaggtataatccaatatttcatatatgtaattcctccacatctcattaaatttttaaattatacacaacctaatttttagtttta

SEQ ID NO: 3 - DNA, 300 bp, Staphylococcus aureus
actactaataatcttaatattcgattgaaaactaattattgtaaggtggaaggtatgaataattatttcatatcttctttttaatattaaaatataatactaaattaataatcatattctttcattaataagctatatataactatcccgcctatttatttaattattaataaaccttacagataaaaaccgctactaaagaggatatggaaatccatctctactttattgttttcttcaaatattatctcgtaatttaccttgttcattaaacaaaaaactggataaaaaaccgcatca

SEQ ID NO: 4 - DNA, 300 bp, Staphylococcus aureus
atttaatgataattttaaaaacatccgaagaaattatccaacaaatctctacatccttatacttgatgaacgatgaaaaaagacagttaatacatgaactcaatacttatatacactattttttaaatagtctacaaaaaattccaaatacagatgcctataaactaacaattacaaattattattttgtgtttcacattataatatatcaactagaattaattcttaataaaaagtaatcattaaaatttaataaactctgctttatattataaaattacggctgaaataaccgcatca

SEQ ID NO: 5 - DNA, 300 bp, Staphylococcus aureus
gtcttagcaaagtgaaattcaaaattttactaaatgaaaaatatgtctatatatgtactttaaaaaatttcattagccgcttatctagtatcgtaatcattcctcacatatttattagcatcttctttacatcgcttactgcaaacatctaatacaaaaagaagtcgatttacacaccatgtattaaataatggaaattcttaatctttacttgtacctaaattatcaaacttaatattcactttttattcttcaaagatttgagctaatttaataattttctcatattttttagtttta

SEQ ID NO: 6 - DNA, 21 bp, Staphylococcus aureus
aaatgaaagactgcggaggct

SEQ ID NO: 7 - DNA, 29 bp, Staphylococcus aureus
atcgtatgatattgcaaggtataatccaa

SEQ ID NO: 8 - DNA, 30 bp, Staphylococcus aureus
ggaaatccatctctactttattgttttctt

SEQ ID NO: 9 - DNA, 20 bp, Staphylococcus aureus
cggctgaaataaccgcatca

SEQ ID NO: 10 - DNA, 30 bp, Staphylococcus aureus
tcgatttacacaccatgtattaaataatgg

SEQ ID NO: 11 - protein, 159 aa, Staphylococcus aureus
MKITILAVGKLKEKYWKQAIAEYEKRLGPYTKIDIIEVPDEKAPENMSDKEIEQVKEKEGQRILAKIKPQSTVITLEIQGKMLSSEGLAQELNQRMTQGQSDFVFVIGGSNGLHKDVLQRSNYALSFSKMTFPHQMMRVVLIEQVYRAFKIMRGEAYHK

SEQ ID NO: 12 - DNA, 480 bp, Staphylococcus aureus
atgaaaatcaccattttagctgtagggaaactaaaagagaaatattggaagcaagccatagcagaatatgaaaaacgtttaggcccatacaccaagatagacatcatagaagttccagacgaaaaagcaccagaaaatatgagcgacaaagaaattgagcaagtaaaagaaaaagaaggccaacgaatactagccaaaatcaaaccacaatccacagtcattacattagaaatacaaggaaagatgctatcttccgaaggattggcccaagaattgaaccaacgcatgacccaagggcaaagcgactttgtattcgtcattggcggatcaaacggcctgcacaaggacgtcttacaacgcagtaactatgcactatcatttagcaaaatgacattcccacatcaaatgatgcgggttgtgttaattgaacaagtgtatagagcatttaagattatgcgtggagaggcgtatcataaataa

SEQ ID NO: 13 - DNA, 20 bp, Staphylococcus aureus
caagggcaaagcgactttgt

SEQ ID NO: 14 - DNA, 22 bp, Staphylococcus aureus
tgctatcttccgaaggattggc

SEQ ID NO: 15 - DNA, 31 bp, Staphylococcus aureus
gtcattacattagaaatacaaggaaagatgc

SEQ ID NO: 16 - DNA, 24 bp, Staphylococcus aureus
cggcctgcacaaggacgtcttaca

(19) United States
(12) Patent Application Publication
(10) Pub. No.: US 2011/0129835 A1
(43) Pub. Date: Jun. 2, 2011

(54) METHODS AND COMPOSITIONS FOR DETECTING METHICILLIN-RESISTANT STAPHYLOCOCCUS AUREUS

(75) Inventor: Pradip Manna, Overland Park, KS (US)

(73) Assignee: PHYSICIANS REFERENCE LABORATORY, LLC, OVERLAND PARK, KS (US)

(21) Appl. No.: 12/956,266

(22) Filed: Nov. 30, 2010

Related U.S. Application Data
(60) Provisional application No. 61/265,153, filed on Nov. 30, 2009.

(51) Int. Cl. C12Q 1/68

(52) U.S. Cl. 435/6.11

(57) ABSTRACT

Provided are methods of detecting the presence and/or amount of methicillin-resistant S. aureus in a sample. The methods may include, in some embodiments, processing the sample with a lysyl endopeptidase followed by amplification and detection of S. aureus chromosomal DNA if present in the sample. The PCR amplification may include the use of one or more forward primers that target SCCmec right chromosomal junction regions, a reverse primer that targets an orfX region and at least one probe that targets and orfX region between the forward and reverse primers. The reverse primer and/or the at least one probe may be targeted to conserved regions of orfX, i.e. regions that do not contain SNPs or other polymorphisms. Sets of oligonucleotides and kits for performing the methods are also provided.

### CROSS-REFERENCE TO RELATED APPLICATIONS

This application claims priority from U.S. Provisional Application Ser. No. 61/265,153 filed on Nov. 30, 2009, incorporated herein by reference in its entirety.

### STATEMENT REGARDING FEDERALLY SPONSORED RESEARCH OR DEVELOPMENT

Not applicable.

### INCORPORATION-BY-REFERENCE OF SEQUENCE LISTING

The Sequence Listing, which is a part of the present disclosure, includes a computer readable file PLAB 100US_ST25.TXT generated by U.S. Patent & Trademark Office PatentIn version 3.5 software comprising nucleotide and/or amino acid sequences of the present invention. The subject matter of the Sequence Listing is incorporated herein by reference in its entirety.

### FIELD

The present teachings relate to microbial detection assays and, more particularly, to methods and compositions for the detection of methicillin-resistant Staphylococcus aureus.

### INTRODUCTION

Staphylococcus aureus (S. aureus) is one of the most significant human pathogens, causing both hospital-acquired and community-acquired infections. S. aureus is often found in the nasal membranes and on the skin of asymptomatic human carriers as well as non-human mammals. Both mild infections, such as those of the skin and soft tissue, as well as severe and life-threatening infections of the blood can be caused by S. aureus.

Methicillin is in the penicillin class of beta-lactam antibiotics. Shortly after methicillin came into clinical use in the early 1960s, strains of methicillin-resistant S. aureus (MRSA) were first detected. Resistance to methicillin is mediated by the presence of penicillin-binding protein 2a (PBP-2a), encoded by the mecA gene. In MRSA, the PBP-2a protein is altered, preventing effective binding of most beta-lactam antibiotics.

Since the end of the 1970's, the occurrence of MRSA has steadily increased. Molecular epidemiological studies have shown that a limited number of MRSA strains have spread by clonal dissemination between different hospitals, cities, countries, and even continents. These MRSA are now the cause of hospital infections worldwide. MRSA strains are most often introduced into an institution through the contaminated hands of an infected or colonized patient or by a colonized health care worker. Thus, epidemiological surveys and control measures are particularly important for the control of MRSA. In addition, rapid screening followed by accurate and timely identification of MRSA can constitute a highly significant component of an institution's preventative measures. Thus there is a continuing need for new approaches for rapid screening and detection of MRSA.

### SUMMARY

Accordingly, the present invention provides new methods and compositions for the detection of MRSA. The methods are based upon an amplification and detection approach that utilizes one or more forward primers that hybridize to an S. aureus SCCmec right extremity junction (MREJ) region, a reverse primer that hybridizes to a first orfX region of S. aureus and at least one probe that hybridizes to a second orfX region between the first orfX region and the MREJ region. In various embodiments, the method may also include sample preparation involving incubating the sample with a lysyl endopeptidase prior to amplification.

Thus, the present invention provides, in various embodiments, methods of detecting the presence and/or amount of MRSA if present in a sample. The methods may include a) processing the sample by contacting the sample with a lysyl endopeptidase to produce a processed sample, b) performing an amplification reaction by contacting the processed sample with a set of primers and at least one probe to produce an amplicon and hybridizing of the at least one probe to the amplicon if the MRSA are present in the sample and c) detecting the hybridizing of the at least one probe to the amplicon as an indication of the presence and/or amount of MRSA in the sample. The set of primers and at least one probe may include a) at least one forward primer that hybridizes to an S. aureus MREJ region, b) a reverse primer that hybridizes to a first S. aureus orfX region and c) at least one probe that hybridizes to a region of the amplicon corresponding to a second S. aureus orfX region. The at least one forward primer may include at least five forward primers. In various embodiments, the lysyl endopeptidase may be a lysyl endopeptidase of Lysobacter sp IB-9374 and, in particular the lysyl endopeptidase may be LepA.

The present invention also provides, in various embodiments, methods of detecting the presence and/or amount of MRSA if present in a sample. The methods may include a) performing an amplification reaction by contacting the sample with a set of forward and reverse primers and at least one probe of not more than 30 nucleotides in length to produce an amplicon and hybridizing of the at least one probe to the amplicon if the MRSA is present in the sample and b) detecting the hybridizing of the at least one probe to the amplicon as an indication of the presence and/or amount of MRSA in the sample. The set of primers may include at least five forward primers that hybridize to an S. aureus MREJ sequence and a reverse primer that hybridizes to a sequence in an orfX region adjacent to the S. aureus MREJ. In various embodiments, the at least one probe may include at least one linear probe such as, for example, a TAQMAN® probe. Further, in various embodiments, the probe may be designed to hybridize to a conserved S. aureus orfX region. In particular, the probe may be designed to hybridize to region having no known single nucleotide polymoprhisms (SNPs). Moreover, in certain embodiments, the at least one probe may include not more than one probe. In various embodiments, the set of primers may include at least five forward primers.

The present invention also provides, in various embodiments, methods of detecting the presence and/or amount of methicillin-resistant S. aureus MRSA if present in a sample. The methods may include a) processing the sample by contacting the sample with a lysyl endopeptidase to produce a processed sample, b) performing an amplification reaction by contacting the processed sample with a set of primers that amplify a target sequence that includes an S. aureus MREJ region and an adjacent orfX region of an MRSA to produce an amplicon and c) detecting the amplicon with at least one probe that hybridizes to the amplified orfX region as an indication of the presence and/or amount of MRSA in the sample. The set of primers may include at least one forward primer that targets an S. aureus MREJ region and a reverse primer that targets an S. aureus orfX region. The at least one forward primmer may include at least five forward primers. In various embodiments, the lysyl endopeptidase may be a lysyl endopeptidase of Lysobacter sp IB-9374 and, in particular the lysyl endopeptidase may be LepA.

The present invention also provides, in various embodiments, methods of detecting the presence and/or amount of MRSA if present in a sample. The methods may include a) performing an amplification reaction by contacting the sample with a set of primers that amplify a target sequence that includes an S. aureus MREJ region and an adjacent orfX region of an MRSA to produce an amplicon and b) detecting the amplicon with at least one probe of not more than 30 contiguous nucleotides that hybridize to the amplified orfX region as an indication of the presence and/or amount of MRSA in the sample. The set of primers may include at least five forward primers and a reverse primer. In various embodiments, the at least one probe may include at least one linear probe such as, for example, a TAQMAN® probe. Further, in various embodiments, the probe may be designed to hybridize to a conserved S. aureus orfX region. In particular, the probe may be designed to hybridize to region having no known single nucleotide polymoprhisms (SNPs).

In various embodiments of each of the methods described above, the at least five forward primers may include a) a forward primer that is targeted to a type II S. aureus MREJ region, b) a forward primer that is targeted to a type III S. aureus MREJ region, c) a forward primer that is targeted to a type IV S. aureus MREJ region, d) a forward primer that is targeted to a type V S. aureus MREJ region, and e) a forward primer that is targeted to a type VII S. aureus MREJ region. In particular, the at least five forward primers may include a first sequence of at least 15 contiguous nucleotides of SEQ ID NO: 1; a second sequence of at least 15 contiguous nucleotides of SEQ ID NO: 2; a third sequence of at least 15 contiguous nucleotides of SEQ ID NO: 3; a fourth sequence of at least 15 contiguous nucleotides of SEQ ID NO: 4; and a fifth sequence of at least 15 contiguous nucleotides of SEQ ID NO: 5. In various embodiments, the at least five forward primers may include SEQ ID NOS: 6-10 or complements thereof.

In various embodiment, the reverse primer may include at least 15 contiguous nucleotides targeted to a nucleotide encoding an orfX polypeptide sequence such as is set forth in SEQ ID NO: 11 or at least 15 contiguous nucleotides of an orfX nucleotide sequence such as is set forth in SEQ ID NO: 12. In particular, the reverse primer may include SEQ ID NO: 13. Further, the at least one probe may include at least 15 contiguous nucleotides targeted to a sequence encoding an orfX polypeptide sequence such as is set forth in SEQ ID NO: 11 or at least 15 contiguous nucleotides of an orfX nucleotide sequence such as is set forth in SEQ ID NO: 12. In particular, the at least one probe may include SEQ ID NO: 14 or a complement thereof.

The present invention also provides, in various embodiments, a set of oligonucleotides for use in a method of detecting the presence and/or amount of MRSA in a sample. The set of oligonucleotides may include a) at least five forward primers, b) a reverse primer that hybridizes to a first S. aureus orfX region and c) at least one probe of not more than 30 contiguous nucleotides in which the probe hybridizes to a second S. aureus orfX region between the first S. aureus orfX region and the MREJ regions. In various embodiments, the at least one probe may include at least one linear probe such as, for example, a TAQMAN® probe. Further, in various embodiments, the probe may be designed to hybridize to a conserved S. aureus orfX region. In particular, the probe may be designed to hybridize to region having no known single nucleotide polymoprhisms (SNPs).

In various embodiments, the at least five forward primers may include sequences that target type II, type III, type IV, type V and type VII S. aureus MREJ regions. In particular, the at least five forward primers may include sequences of at least 15 contiguous nucleotides of each of SEQ ID NOS: 1-5. In various embodiments, at least five forward primers may include SEQ ID NOS: 6-10 or complements thereof. In various embodiments, the reverse primer may include at least 15 contiguous nucleotides that target a sequence encoding an orfX polypeptide sequence such as is set forth in SEQ ID NO: 11 or at least 15 contiguous nucleotides that target a nucleotide sequence of an orfX such as is set forth in SEQ ID NO: 12. In particular, the reverse primer may include SEQ ID NO: 13 or a complement thereof. Further, the at least one probe may include at least 15 contiguous nucleotides targeted to a sequence encoding an orfX polypeptide sequence such as is set forth in SEQ ID NO: 11 or at least 15 contiguous nucleotides that target a nucleotide sequence of an orfX such as is set forth in SEQ ID NO: 12. In particular, the at least one probe may include SEQ ID NO: 14 or a complement thereof.

The present invention also provides, in various embodiments, kits for detecting the presence and/or amount of MRSA if present in a sample. The kits may include a) a lysyl endopeptidase; b) at least one forward primer that hybridizes to an S. aureus MREJ region; c) a reverse primer that hybridizes to a first orfX region; and d) at least one probe that hybridizes to a second S. aureus orfX region between the first S. aureus orfX region and the MREJ region. The at least one forward primer may be at least five forward primers. The kits may further include reagents suitable for performing a PCR reaction that produces an amplicon and hybridizing of the at least one probe to the amplicon if the MRSA is present in the sample. In various embodiments, the lysyl endopeptidase may be a lysyl endopeptidase of Lysobacter sp IB-9374 and, in particular the lysyl endopeptidase may be LepA.

In still other embodiments, the present invention provides kits for detecting the presence and/or amount of MRSA if present in a sample. The kits may include a) at least five forward primers, b) a reverse primer that hybridizes to a first orfX region and c) at least one probe of not more than 30 contiguous nucleotides that hybridize to a second S. aureus orfX region between the first S. aureus orfX region and the MREJ region. The kits may further include reagents suitable for performing a PCR reaction that produces an amplicon and hybridizing of the at least one probe to the amplicon if the MRSA is present in the sample. In various embodiments, the at least one probe may include at least one linear probe such as, for example, a TAQMAN® probe. Further, in various embodiments, the probe may be designed to hybridize to a conserved S. aureus orfX region. In particular, the probe may be designed to hybridize to region having no known single nucleotide polymoprhisms (SNPs).

In various embodiments, the kits described above may include at least five forward primers that include sequences that target type II, type III, type IV, type V and type VII S. aureus MREJ regions. In particular, the at least five forward primers may include sequences of at least 15 contiguous nucleotides of each of SEQ ID NOS: 1-5. In various embodiments, at least five forward primers may include SEQ ID NOS: 6-10 or complements thereof. In various embodiments, the reverse primer may include at least 15 contiguous nucleotides that target a sequence encoding an orfX polypeptide sequence such as is set forth in SEQ ID NO: 11 or at least 15 contiguous nucleotides that target a nucleotide sequence of an orfX such as is set forth in SEQ ID NO: 12. In particular, the reverse primer may include SEQ ID NO: 13 or a complement thereof. Further, the at least one probe may include at least 15 contiguous nucleotides targeted to a sequence encoding an orfX polypeptide sequence such as is set forth in SEQ ID NO: 11 or at least 15 contiguous nucleotides that target a nucleotide sequence of an orfX such as is set forth in SEQ ID NO: 12. In particular, the at least one probe may include SEQ ID NO: 14 or a complement thereof.

### DRAWINGS

Not applicable.

### DETAILED DESCRIPTION

### Abbreviations and Definitions

To facilitate understanding of the invention, a number of terms and abbreviations as used herein are defined below as follows:

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Bind, Binds or Interacts With: As used herein, “bind,” “binds,” or “interacts with” means that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ moles/liter for that second molecule.

Coding sequence: The coding sequence that encodes any known or putative protein or polypeptide such as, for example, orfX. An orfX coding sequence may be identical to a sequence provided herein, or it may be a different coding sequence as a result of the redundancy/degeneracy of the genetic code. Nevertheless an orfX sequence encodes the same putative polypeptide as the polynucleotides described herein. Examples of nucleotide codons of S. aureus are summarized in Table 1, below:

TABLE 1
S. aureus Nucleotide Codons.

| | | Abbreviation | Abbreviation |
|---|---|---|---|
| Codon | Full Name | (3 Letter) | (1 Letter) |
| TTT | Phenylalanine | Phe | F |
| TTC | Phenylalanine | Phe | F |
| TTA | Leucine | Leu | L |
| TTG | Leucine | Leu | L |
| TCT | Serine | Ser | S |
| TCC | Serine | Ser | S |
| TCA | Serine | Ser | S |
| TCG | Serine | Ser | S |
| TAT | Tyrosine | Tyr | Y |
| TAC | Tyrosine | Tyr | Y |
| TAA | Termination | Ter | X |
| TAG | Termination | Ter | X |
| TGT | Cysteine | Cys | C |
| TGC | Cysteine | Cys | C |
| TGA | Termination | Ter | X |
| TGG | Tryptophan | Trp | W |
| CTT | Leucine | Leu | L |
| CTC | Leucine | Leu | L |
| CTA | Leucine | Leu | L |
| CTG | Leucine | Leu | L |
| CCT | Proline | Pro | P |
| CCC | Proline | Pro | P |
| CCA | Proline | Pro | P |
| CCG | Proline | Pro | P |
| CAT | Histidine | His | H |
| CAC | Histidine | His | H |
| CAA | Glutamine | Gln | Q |
| CAG | Glutamine | Gln | Q |
| CGT | Arginine | Arg | R |
| CGC | Arginine | Arg | R |
| CGA | Arginine | Arg | R |
| CGG | Arginine | Arg | R |
| ATT | Isoleucine | Ile | I |
| ATC | Isoleucine | Ile | I |
| ATA | Isoleucine | Ile | I |
| ATG | Methionine | Met | M |
| ACT | Threonine | Thr | T |
| ACC | Threonine | Thr | T |
| ACA | Threonine | Thr | T |
| ACG | Threonine | Thr | T |
| AAT | Asparagine | Asn | N |
| AAC | Asparagine | Asn | N |
| AAA | Lysine | Lys | K |
| AAG | Lysine | Lys | K |
| AGT | Serine | Ser | S |
| AGC | Serine | Ser | S |
| AGA | Arginine | Arg | R |
| AGG | Arginine | Arg | R |
| GTT | Valine | Val | V |
| GTC | Valine | Val | V |
| GTA | Valine | Val | V |
| GTG | Valine | Val | V |
| GCT | Alanine | Ala | A |
| GCC | Alanine | Ala | A |
| GCA | Alanine | Ala | A |
| GCG | Alanine | Ala | A |
| GAT | Aspartate | Asp | D |
| GAC | Aspartate | Asp | D |
| GAA | Glutamate | Glu | E |
| GAG | Glutamate | Glu | E |
| GGT | Glycine | Gly | G |
| GGC | Glycine | Gly | G |
| GGA | Glycine | Gly | G |
| GGG | Glycine | Gly | G |

Complementary: As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions, e.g., stringent conditions, with an oligonucleotide or polynucleotide comprising the second nucleotide sequence, as will be understood by the skilled person. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides. This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a DNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes of the invention. “Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled.

The terms “complementary”, “fully complementary” and “substantially complementary” herein may be used with respect to the base matching between the two complementary strands of double stranded chromosomal DNA, or between the sense and antisense strands of a coding region of chromosomal DNA such as orfX or between a DNA primer or probe and a target sense or antisense sequence, as will be understood from the context of their use. As used herein, a polynucleotide which is “substantially complementary to at least part of a target nucleic acid refers to a polynucleotide that is substantially complementary to a contiguous portion of the DNA of interest.

Conserved orfX region: The term “conserved orfX region” as used herein refers a region of the orfX sequence that is identical in all known S. aureus. The term is intended to include, but not be limited to, regions of an orfX sequence that do not contain any single nucleotide polymorphisms (SNPs) or other polymorphisms such as two or more nucleotide polymorphisms in any known S. aureus. Thus, a conserved orfX region will be present in all MRSA that have been identified and whose orfX sequences at least in the conserved region, are known and available in a database such as, for example, GenBank. The GenBank database is searchable online at the internet site http://www.ncbi.nlm.nih.gov/. Other databases may include EMBL Nucleotide Sequence Database and DNA Data Bank of Japan (DDBJ).

Conservative Amino Acid Changes: As used herein, when referring to mutations in a nucleic acid molecule, “conservative changes” are those in which at least one codon in the protein-coding region of the nucleic acid has been changed such that at least one amino acid of the polypeptide encoded by the nucleic acid sequence is substituted with a another amino acid having similar characteristics. Examples of conservative amino acid substitutions are ser for ala, thr, or cys; lys for arg; gln for asn, his, or lys; his for asn; glu for asp or lys; asn for his or gln; asp for glu; pro for gly; leu for ile, phe, met, or val; val for ile or leu; ile for leu, met, or val; arg for lys; met for phe; tyr for phe or trp; thr for ser; trp for tyr; and phe for tyr.

Detection probe: The term “detection probe” refers to a molecule that hybridizes to a target nucleic acid molecule and provides detectability for the presence of the target molecule. Such probes may include TAQMAN® probes, Molecular Beacons, Scorpions, FRET probes and the like. The detection probe may be specific for a particular MRSA type, for more than one MRSA type or for S. aureus in general, i.e. for MSSA and MRSA. The detection probe may be labeled, for example, with a fluorescent, luminescent or enzymatic label which provides the means for ascertaining the presence of the target molecule. A fluorogenic probe is a probe that contains a fluorescent moiety such as, for example, a TAQMAN® probe. In various embodiments, probes such as TAQMAN® probes may contain a reporter dye attached to the 5′ end and a quencher dye attached to the 3′ end. Reporter and quencher substances for use in probes are known. The term “linear probe” as used herein is intended to include probes such as TAQMAN® probes that do not have self-complementary regions that become self-hybridized and such linear probes do not form stem-loop configurations such as occurs, for example, with molecular beacon probes. Further, a detection probe of the invention may be a DNA molecule or a Peptide Nucleic Acid (PNA) molecule (see, for example, U.S. Pat. No. 5,539,082, Egholm et al., Nature 365:566-568, 1993; U.S. Patent Application Publication 20090246758).

Probe design may involve selection parameters that may include, but are not limited to, one or more of melting temperatures of the primer pairs and the length of the probes, the G-C content of the probes. In various embodiments, the length of a probe may be at least about 10, at least about 15, at least about 20, at least about 25, at least about 30, at least about 35 or at least about 40 contiguous nucleotides and, in particular, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, contiguous nucleotides and, more particularly, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more contiguous nucleotides. In various embodiments, the length of a probe may be not more than about 40, not more than about 35, not more than about 30, not more than about 25, not more than about 20 or not more than about 15 contiguous nucleotides and, in particular, not more than 40, not more than 39, not more than 38, not more than 37, not more than 36, not more than 35, not more than 34, not more than 33, not more than 32, not more than 31, not more than 30, not more than 29, not more than 28, not more than 27, not more than 26, not more than 25, not more than 24, not more than 23, not more than 22, not more than 21, not more than 20, not more than 19, not more than 18, not more than 17, not more than 16 or not more than 15 contiguous nucleotides.

DNA Amplification: The term “DNA amplification” refers to the production of multiple copies of a sequence of a DNA. DNA amplification technology or DNA amplification procedures or DNA amplification reactions may include polymerase chain reaction (PCR), ligase chain reaction (LCR), nucleic acid sequence-based amplification (NASBA), self-sustained sequence replication (3SR), strand displacement amplification (SDA), branched DNA signal amplification (bDNA), transcription-mediated amplification (TMA), cycling probe technology (CPT), nested PCR, multiplex PCR, solid phase amplification (SPA), nuclease dependent signal amplification (NDSA), rolling circle amplification technology (RCA), anchored strand displacement amplification, solid-phase (immobilized) rolling circle amplification, Q Beta replicase amplification and the like (see, for example, Schweitzer and Kingsmore, Current Opinion in Biotechnology 12: 21-27, 2001; Kawashima et al., PCT publication WO/2004/016755; DNA Amplification: Current Technologies and Applications, Demidov and Broude, Eds., Horizon Bioscience, Norfolk, UK, 2004). For example, DNA amplification by PCR involves thermal cycling to produce repeated cycles of denaturation, the annealing of primers to DNA complementary to the primers followed by primer extension.

Primer design may involve selection parameters that may include, but are not limited to, one or more of melting temperatures of the primer pairs, length of the primers or probes, the G-C content of the primers and the size of the amplicon product. In various embodiments, the length of primers may be at least about 15, at least about 20, at least about 25, at least about 30, at least about 35 or at least about 40 contiguous nucleotides and, in particular, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, contiguous nucleotides and, more particularly, 15, 16, 17, 18, 19, 20, 21, 22, 23, 24, 25, 26, 27, 28, 29, 30, 31, 32, 33, 34, 35, 36, 37, 38, 39, 40 or more contiguous nucleotides. In various embodiments, the length primers may be not more than about not more than about 40, not more than about 35, not more than about 30, not more than about 25, not more than about 20 or not more than about 15 contiguous nucleotides and, in particular, not more than 40, not more than 39, not more than 38, not more than 37, not more than 36, not more than 35, not more than 34, not more than 33, not more than 32, not more than 31, not more than 30, not more than 29, not more than 28, not more than 27, not more than 26, not more than 25, not more than 24, not more than 23, not more than 22, not more than 21, not more than 20, not more than 19, not more than 18, not more than 17, not more than 16 or not more than 15 contiguous nucleotides.

DNA detection: DNA detection technology or DNA detection procedures or DNA detection reactions may include any nucleic acid detection method. Such methods are well known in the art and may involve the use of labeled detection probes. Methods that use fluorescent probes for nucleic acid detection may be based upon a hybridization-triggered fluorescence of intact probes (e.g. methods using molecular beacons) or based upon a quenched-fluorescence release of a probe digested by DNA Polymerase (e.g., methods using TAQMAN® probes). Nucleic acid hybridization techniques and conditions are known to the skilled artisan and have been described for example, in Sambrook et al. Molecular Cloning A Laboratory Manual, 2nd Ed. Cold Spring Lab. Press, December 1989.

Fragment: A “fragment” of a nucleic acid is a portion of a nucleic acid that is less than full-length and comprises at least a minimum length capable of hybridizing specifically with a native nucleic acid under stringent hybridization conditions. The length of such a fragment may be at least 15 nucleotides, at least 20 nucleotides or at least 30 nucleotides. A “fragment” of a polypeptide is a portion of a polypeptide that is less than full-length (e.g., a polypeptide consisting of 5, 10, 15, 20, 30, 40, 50, 75, 100 or more amino acids of a native protein), and preferably retains at least one functional activity of a native protein.

Functional Activity: As used herein, the term “functional activity” refers to a protein having any activity associated with the physiological function of the protein.

Gene: As used herein, the term “gene” means a nucleic acid molecule that codes for a particular protein.

Hybridizing: The term “hybridization” refers to any process by which a strand of nucleic acid or a PNA binds with a complementary strand of nucleic acid through base pairing. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.” Reference to hybridizing to an MREJ sequence or an orfX sequence of double stranded chromosomal DNA means hybridizing to either strand including either the sense or antisense strand of a coding sequence. This may also be indicated herein by reference to a primer or probe having a particular sequence or the complement thereof or by reference to hybridization to a particular sequence or the complement thereof.

Homolog: As used herein, the term “homolog” refers to a target gene encoding a target polypeptide isolated from an organism other than a human being.

Labeled: The term “labeled,” with regard to a probe, is intended to encompass direct labeling of the probe by coupling (i.e., physically linking) a detectable substance to the probe.

Lysyl endopeptidase: Lysyl endopeptidase is an enzyme that hydrolyzes lysyl bonds. Such enzymes have been identified in Achromobacter lyticus 497-1, Lysobacter enzymogenes, Pseudomonas aeruginosa, and more recently in lysobacter sp. Strain IB-9374 (Ahmed et al., J Bioscience Bioengineering 95:27-34, 2003; Chohnan et al, FEMS Microbiol Lett 213:13-20, 2002). The lysobacter sp IB-9374 enzyme is a ˜27 kDa protein that has been referenced as achromopeptidase and as LepA (see GenBank Accession No. AB045676.1 for precursor polypeptide and nucleic acid sequence and Accession No. BAB32450.1 for mature protein sequence). A second lysyl endopeptidase, LepB, has also been isolated from lysobacter sp IB-9374 (Chohnan et al., J Bacteriol 186: 5093-5100, 2004).

Methicillin-resistant S. aureus (MRSA): MRSA are S. aureus that have acquired the exogenous gene, mecA, that encodes a penicillin-binding protein, 2a (PBP-2a) with decreased affinity for β-lactam antibiotics. (for reviews see Gordon and Lowy, Clin Infect Dis 46 (Suppl 5) S350-359, 2008; Malhotra-Kumar et al., Journal of Clinical Microbiology 46:1577-1587; 2008; Ito et al., Methods Mol Biol 391:87-102, 2007). The mecA gene is carried by a mobile genetic element, the staphylococcal cassette chromosome mec (SCCmec) which is inserted near the chromosomal origin of replication. SCCmec elements carry the mec and the ccr gene complexes and the elements further contain both inverted and direct repeats at both ends. The SCCmec DNAs are integrated at a specific site in the methicillin-susceptible S. aureus chromosome which is located at the 3′ end of an open reading frame, orfX, of unknown function. The MRSA have been classified in five phenotypes based upon the mec and ccr class of the MRSA. An alternative classification has been based upon the polymorphic genotypes of the right extremity junction of the SCCmec. MRSA types referenced herein are based upon the latter classification, i.e. the genotypes of the SCCmec right extremity junction (MREJ). Examples of GenBank accession numbers for the MREJ types are as follows: Type II MREJ is exemplified by Accession No. DQ106887; Type III MREJ is exemplified by Accession No. AF422696; Type IV Accession No. is exemplified by Accession NO. AY267374; Type V MREJ is exemplified by Accession No. AY267381 and TypeVII MREJ is exemplified by Accession No. AY7384.1.

Methicillin-susceptible S. aureus (MSSA): MSSA are S. aureus that have not acquired the mecA gene and, as a result, remain susceptible to β-lactam antibiotics.

Native: When referring to a nucleic acid molecule or polypeptide, the term “native” refers to a naturally-occurring (e.g., a “wild-type”) nucleic acid or polypeptide.

Nucleic Acid or Nucleic Acid Molecule: As used herein, the term “nucleic acid” or “nucleic acid molecule” means a chain of two or more nucleotides such as RNA (ribonucleic acid) and DNA (deoxyribonucleic acid). A “purified” nucleic acid molecule is one that is substantially separated from other nucleic acid sequences in a cell or organism in which the nucleic acid naturally occurs (e.g., 30%, 40%, 50%, 60%, 70%, 80%, 90%, 95%, 96%, 97%, 98%, 99% or 100% free of contaminants). The term includes, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced polymerase chain reaction (PCR), nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

Protein or Polypeptide: As used herein, “protein” or “polypeptide” mean any peptide-linked chain of amino acids, regardless of length or post-translational modification, e.g., glycosylation or phosphorylation. A “purified” polypeptide is one that is substantially separated from other polypeptides in a cell or organism in which the polypeptide naturally occurs (e.g., 30%, 40%, 50%, 60%, 70%, 80%, 90%, 95%, 96%, 97%, 98%, 99% or 100% free of contaminants).

Purified substance: A “purified” substance is one that is substantially separated from other undesired substances such as contaminants that may naturally occur with the substance (e.g., 30%, 40%, 50%, 60%, 70%, 80%, 90%, 95%, 96%, 97%, 98%, 99% or 100% free of contaminants).

Sense Strand: The term “sense strand,” as used herein, refers to the strand of a double stranded chromosomal DNA that includes a coding region that is substantially complementary to a region of the antisense strand.

Sequence Identity: As used herein, “sequence identity” means the percentage of identical subunits at corresponding positions in two sequences when the two sequences are aligned to maximize subunit matching, i.e., taking into account gaps and insertions. Sequence identity is present when a subunit position in both of the two sequences is occupied by the same nucleotide or amino acid, e.g., if a given position is occupied by an adenine in each of two DNA molecules, then the molecules are identical at that position. For example, if 9 positions in a sequence 10 nucleotides in length are identical to the corresponding positions in a second 10-nucleotide sequence, then the two sequences have 90% sequence identity. Percent sequence identity of an antisense compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Silent and Conservative: When referring to mutations in a nucleic acid molecule, “silent” changes are those that substitute of one or more base pairs in the nucleotide sequence, but do not change the amino acid sequence of the polypeptide encoded by the sequence. “Conservative” changes are those in which at least one codon in the protein-coding region of the nucleic acid has been changed such that at least one amino acid of the polypeptide encoded by the nucleic acid sequence is substituted with a another amino acid having similar characteristics. Examples of conservative amino acid substitutions are ser for ala, thr, or cys; lys for arg; gln for asn, his, or lys; his for asn; glu for asp or lys; asn for his or gln; asp for glu; pro for gly; leu for ile, phe, met, or val; val for ile or leu; ile for leu, met, or val; arg for lys; met for phe; tyr for phe or trp; thr for ser; trp for tyr; and phe for tyr.

Single Nucleotide Polymorphism: As used herein, the term “single nucleotide polymorphism” or “SNP” refers to any position along a nucleotide sequence that has one or more variant nucleotides. SNPs occurring in a coding region in which both forms code for the same polypeptide sequence due to code degeneracy are termed “synonymous SNPs.” In such instances the SNP occurs in the third base of a codon that encodes a particular amino acid of the polypeptide. SNPS that result in a different polypeptide sequence are termed “nonsynonymous SNPs.” With respect to the orfX region of MRSA and MSSA, it is known that synonymous SNPs occur, i.e. changes in the third base of certain codons in the orfX region that do not alter the putative sequence of the deduced polypeptide. Regions of the orfX sequence that are not known to contain such SNPs are referenced herein as conserved regions of the orfX sequence.

Strand Comprising a Sequence: As used herein, the term “strand comprising a sequence” refers to an oligonucleotide comprising a chain of nucleotides that is described by the sequence referred to using the standard nucleotide nomenclature.

Stringent Hybridization Conditions or Stringent Conditions: As used herein, the term “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomeric compounds and the assays in which they are being investigated. For example, hybridization conducted under “low stringency conditions” means in 10% formamide, 5×Denhart's solution, 6×SSPE, 0.2% SDS at 42° C., followed by washing in lx SSPE, 0.2% SDS, at 50° C.; “moderate stringency conditions” means in 50% formamide, 5×Denhart's solution, 5×SSPE, 0.2% SDS at 42° C., followed by washing in 0.2×SSPE, 0.2% SDS, at 65° C.; and “high stringency conditions” means in 50% formamide, 5×Denhart's solution, 5×SSPE, 0.2% SDS at 42° C., followed by washing in 0.1×SSPE, and 0.1% SDS at 65° C.

Sample: As used herein, the term “sample” may include but is not limited to: any clinical sample, environmental sample, microbial culture or microbial colony, tissue or cell line. Such clinical samples may include but are not limited to a swab of an infected wound, a skin swab, a nasal swab, a throat swab, a groin swab, an axillary swab, a perineum swab, a swab from a site of an invasive device, a swab, a body fluid, a blood sample or a urine sample. In various embodiments, the term “sample” may refer to a sample that has been processed to release DNA present in any MRSA or MSSA in the original sample. Such processing may include lysis of the MRSA or MSSA.

Subject: As used herein, the terms “subject” and “subjects” refer to any mammal, including a human mammal. Human subjects include any human from whom a sample may be taken or has been taken.

Non-human animal subjects may include, but are not limited to, mammals such as primates, mice, pigs, cows, cats, goats, rabbits, rats, guinea pigs, hamsters, horses, sheep, dogs, and the like. Such animals may be companion animals, as in the case of dogs and cats, for example, or may be trained animals including therapy animals such as a therapy dog. Also included are service animals, such as dogs that assist persons who are in need of assistance due to loss or impairment of sight, hearing, or other senses. Further, non-human subjects may include working animals such as dogs or other animals trained for security or rescue work. Also included are animals trained or maintained for procreation or entertainment purposes, including purebred animal breeds, racehorses, or workhorses. Animals that are genetically-engineered are likewise included, regardless of the purposes of the genetic engineering, as are rare or exotic animals, including zoo animals and wild animals.

Target Sequence: As used herein, “target sequence” refers to a contiguous portion of a nucleic acid sequence, in particular, a DNA sequence. The DNA sequence may be a chromosomal DNA, for example, of S. aureus such as an MRSA or a MSSA. The term “target sequence” means a DNA sequence that is targeted by virtue of complementarity of the DNA sequence to another sequence such as a primer or a probe of the invention. The target DNA sequence may be either strand of double stranded chromosomal DNA of an S. aureus and/or the target DNA sequence may be the sense strand or the antisense strand of a coding region. The target sequence with respect to a set of forward and reverse primers of a DNA amplification reaction refers to the portion of the DNA sequence that is to be amplified in a DNA amplification reaction. The target sequence of a probe in a detection reaction refers to the portion of the DNA sequence to which the probe binds in the detection reaction. Reference to hybridization of a primer or probe to a target sequence of a chromosomal DNA may include hybridization to either of the strands of double stranded chromosomal DNA. This may also be indicated herein by reference to a primer or probe having a particular sequence or the complement thereof or by reference to hybridization to a sequence or the complement thereof.

“G,” “C,” “A”, “T” and “U” (irrespective of whether written in capital or small letters) each generally stand for a nucleotide that contains guanine, cytosine, adenine, thymine, and uracil as a base, respectively. However, it will be understood that the term “nucleotide” can also refer to a modified nucleotide or a surrogate replacement moiety. The skilled person is well aware that guanine, cytosine, adenine and thymine may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine.

### Methods of Detecting MRSA

The present invention provides methods of detecting MRSA based upon amplification and detection of chromosomal DNA of MRSA. The methods involve providing a sample suspected of containing MRSA and then testing the sample for the presence of chromosomal DNA of MRSA. The sample may be a sample that contains chromosomal DNA obtained from intact cells or the sample may be an original sample obtained from a subject or other source such that pre-processing of the sample such as by lysis of any intact cells and/or sample purification, is performed prior to testing. The methods utilize one or more forward primers that target an S. aureus SCCmec right extremity junction (MREJ) region, a reverse primer that targets a first orfX region of S. aureus and a probe that targets a region between the first orfX region and the MREJ region. In various embodiments, the method may also include pre-processing the sample using a lysyl endopeptidase.

### Pre-Processing of Samples

Prior to performing amplification and detection steps in the detection method of the invention, sample preparation is performed which involves lysis of bacterial cells to release chromosomal DNA and processing the sample to remove or inactivate contaminants. Vortexing the sample in the presence of microbeads along with thermal lysis at 95° C. has been used to prepare samples of MRSA for PCR (see for example, BD GeneOhm MRSA Assay package insert, BD Diagnostics, 2009 available online at the internet site http://www.bd.com/geneohm/english/products/pdfs/mrsa_pkginsert.pdf). Typically, lysozymes have not been used for lysis of S. aureus which is known to be lysozyme resistant (Bera et al., Molecular Microbiology 55:778-787, 2004; Bera et al., Infection and Immunity 74:4598-4604, 2006).

In various embodiments, the present invention provides for the treatment of samples with a lysyl endopeptidase in sample preparation which substantially reduces the number of unresolved samples following testing. Unresolved samples refers to samples that cannot be classified as either MRSA positive or MRSA negative upon testing. The inability to resolve samples has been reported for the BD GeneOhm MRSA assay at a level of about 35/778 or 4.5% (BD GeneOhm MRSA Assay package insert, BD Diagnostics, 2009). The present studies have observed a substantial number of unresolved samples with the BD GeneOhm MRSA assay (13%), whereas, the assay methods of the present invention using pretreatment with lysyl endopeptidase produced substantially no unresolved samples (see Example 2 below).

Any lysyl endopeptidase may be used in the present invention. Such lysyl endopeptidases are enzymes that hydrolyze lysyl bonds. Lysyl endopeptidases of the present invention may be obtained from Lysobacter sp., in particular, lysobacter sp. Strain IB-9374 (see, for example, Ahmed et al., J Bioscience Bioengineering 95:27-34, 2003; Chohnan et al, FEMS Microbiol Lett 213:13-20, 2002). The lysobacter sp IB-9374 enzyme is a ˜27 kDa protein that has been referenced as achromopeptidase and as LepA. Other lysyl endopepdidases may also be used in the present invention including a second lysyl endopeptidase, LepB identified in lysobacter sp IB-9374 (Chohnan et al, J Bacteriology 186:5093-5100, 2004) as well as lysyl endopeptidases identified in Achromobacter lyticus 497-1, Lysobacter enzymogenes and Pseudomonas aeruginosa (Ahmed et al., J Bioscience Bioengineering 95:27-34, 2003).

### DNA Amplification and Detection

Amplification of chromosomal DNA of MRSA in accordance with the present invention may be achieved by any of a variety of DNA amplification methods. In particular, the DNA amplification may be performed using PCR methods. DNA amplification by PCR involves the binding of two oligonucleotide primers to target sequences of each of the two strands of the heat-denatured double-stranded chromosomal DNA from the S. aureus to be detected in the sample. Exponential amplification of the target DNA segments is achieved by successive thermal cycles that repeatedly denature the DNA, anneal the primers to the DNA target segments and synthesize new target segments at each cycle. Detection of the amplified DNA target segment is achieved with a detection probe, typically a fluorogenic detection probe. Any real time or post-amplification technology may be used for detection of amplified DNA. Real time PCR may advantageously be used such as, for example, that described in WO 97/46707, WO 97/46712 or WO 97/46714.

The methods of the present invention utilize sets of oligonucleotides that include at least one forward primer targeted to a MREJ region of MRSA, a reverse primer targeted to an orfX sequence in a region adjacent to the MREJ region and a probe that binds to an orfX sequence that lies between the binding sites of the forward and reverse primers. In various embodiments, the forward and reverse primers of the invention may independently comprise or consist of sequences of at least 10 nucleotides, at least 15 nucleotides or at least 20 nucleotides. In various embodiments, the forward and reverse primers may independently comprise or consist of sequences of from about 15 to about 40, from about 15 to about 35 or from about 20 to about 30 contiguous nucleotides in length and in particular, 15, 16, 17, 18, 19, 20, 21, 22, 23, 24, 25, 26, 27, 28, 29, 30, 31, 32, 33, 34, 35, 36, 37, 38, 39 or 40 contiguous nucleotides in length.

In various embodiments, the at least one forward primer may be at least 1, at least 2, at least 3, at least 4, at least 5, at least 6, at least 7, at least 8 or more forward primers and, in particular, 1, 2, 3, 4, 5, 6, 7, 8 or more forward primers. Each of the at least one forward primers may be targeted to at least one MREJ genotype so as to provide for detection of a substantial percentage of known MRSA in various embodiments, and, in some embodiments, substantially all known MRSA. Thus, the detection method of the invention can detect at least about 60%, at least about 65%, at least about 70%, at least about 75%, at least about 80%, at least about 85%, at least about 90%, at least about 95%, at least about 98% or at least about 100% of known MRSA. By known MRSA, it is meant that the MRSA has been sequenced at least to an extent sufficient for MREJ genotyping and the determined sequence is provided in a database such as, for example, GenBank NCBI database. Other databases may include EMBL Nucleotide Sequence Database and DNA Data Bank of Japan (DDBJ). It is contemplated that as new and different MREJ genotypes become known, one or more additional forward primers may be added to the set of primers of the present invention.

In various embodiments, the at least one forward primer may be at least one, at least two, at least three, at least four or at least five forward primers each of which may be targeted to a different one of a Type II MREJ, a Type III MREJ, a Type IV MREJ, a Type V MREJ or a Type VII MREJ such as, for example, the sequences set forth in Table 2 or complements of sequences in the table. In various embodiments, the at least one forward primer may be at least one, at least two, at least three, at least four or at least five forward primers each of which may include a sequence of contiguous nucleotides of a different one of a Type II MREJ, a Type III MREJ, a Type IV MREJ, a Type V MREJ or a Type VII MREJ such as, for example, the sequences as set forth in SEQ ID NOS: 1-5 in Table 2 or complements of sequences in the table.

TABLE 2
MERJ Sequences

| SEQ ID NO | DESCRIPTION | SEQUENCE |
|---|---|---|
| 1 | Type II MERJ; | tatattatgt ctcattttct tcaatatgta cttatttata |
| | Accession No. | ttttaccgta atttactata tttagttgca gaaagaattt |
| | DQ106887; | tctcaaagct agaactttgc ttcactataa gtattcagta |
| | Nucleotides | taaagaatat ttcgctatta tttacttgaa atgaaagact |
| | 22415-22714 | gcggaggcta actatgtcaa aaatcatgaa cctcattact |
| | | tatgataagc ttcttaaaaa cataacagca attcacataa |
| | | acctcatatg ttctgataca ttcaaaatcc ctttatgaag |
| | | cggctgaaaa aaccgcatca |
| 2 | Type III MERJ; | aatctaataa acttcctttt attaatcgta ggcattgtat |
| | Accession No. | atttcctttc attctttctt gattccatta gtttaaattt |
| | AF422696; | aaaatttcat catcaatttc ttaatttaat tgtagttcca |
| | Nucleotides | taatcaatat aatttgtaca gttattatat attctagatc |
| | 8797-9096 | atcaatagtt gaaaaatggt ttattaaaca ctctataaac |
| | | atcgtatgat attgcaaggt ataatccaat atttcatata |
| | | tgtaattcct ccacatctca ttaaattttt aaattataca |
| | | caacctaatt tttagtttta |
| 3 | Type IV MERJ; | Actactaata atcttaatat tcgattgaaa actaattatt |
| | Accession No. | gtaaggtgga aggtatgaat aattatttca tatcttcttt |
| | AY267374 | ttaatattaa aatataatac taaattaata atcatattct |
| | Complement of | ttcattaata agctatatat aactatcccg cctatttatt |
| | Nucleotides | taattattaa taaaccttac agataaaaac cgctactaaa |
| | 456-755 | gaggatatgg aaatccatct ctactttatt gttttcttca |
| | | aatattatct cgtaatttac cttgttcatt aaacaaaaaa |
| | | ctggataaaa aaccgcatca |
| 4 | Type V MERJ; | atttaatgat aattttaaaa acatccgaag aaattatcca |
| | Accession No. | acaaatctct acatccttat acttgatgaa cgatgaaaaa |
| | AY267381; | agacagttaa tacatgaact caatacttat atacactatt |
| | Complement of | ttttaaatag tctacaaaaa attccaaata cagatgccta |
| | Nucleotides | taaactaaca attacaaatt attattttgt gtttcacatt |
| | 448-747 | ataatatatc aactagaatt aattcttaat aaaaagtaat |
| | | cattaaaatt taataaactc tgctttatat tataaaatta |
| | | cggctgaaat aaccgcatca |
| 5 | Type VII MERJ: | gtcttagcaa agtgaaattc aaaattttac taaatgaaaa |
| | Accession No. | atatgtctat atatgtactt taaaaaattt cattagccgc |
| | AY267384.1 | ttatctagta tcgtaatcat tcctcacata tttattagca |
| | Complement of | tcttctttac atcgcttact gcaaacatct aatacaaaaa |
| | Nucleotides | gaagtcgatt tacacaccat gtattaaata atggaaattc |
| | 469-768 | ttaatcttta cttgtaccta aattatcaaa cttaatattc |
| | | actttttatt cttcaaagat ttgagctaat ttaataattt |
| | | tctcatattt tttagtttta |

In various embodiments, the at least one forward primer may comprise or consist of sequences as set forth in any one, two three, four or five of SEQ ID NOS: 6-10 as follows or a complement thereof:

SEQ ID NO: 6
(Type II MERJ Forward Primer,
AAATGAAAGACTGCGGAGGCT);
SEQ ID NO: 7
(Type III MERJ Forward Primer,
ATCGTATGATATTGCAAGGTATAATCCAA);
SEQ ID NO: 8
(Type IV MERJ Forward Primer,
GGAAATCCATCTCTACTTTATTGTTTTCTT);
SEQ ID NO: 9
(Type V MERJ Forward Primer,
CGGCTGAAATAACCGCATCA);
SEQ ID NO: 10
(Type VII MERJ Forward Primer,
TCGATTTACACACCATGTATTAAATAATGG).

In various embodiments, the reverse primer may include a sequence targeted to an orfX region adjacent to the MREJ region such as a nucleotide sequence encoding an orfX polypeptide or an orfX nucleotide sequence or a complement thereof. An example of one orfX nucleotide and putative polypeptide sequence is set forth in Table 3 below.

TABLE 3
Nucleotide and Putative Polypeptide Sequence of orfX

| SEQ ID NO | DESCRIPTION | SEQUENCE |
|---|---|---|
| 11 | S. aureus orfX | MKITILAVGK LKEKYWKQAI AEYEKRLGPY TKIDIIEVPD |
| | Accession No. | EKAPENMSDK EIEQVKEKEG QRILAKIKPQ STVITLEIQG |
| | AB121219 | KMLSSEGLAQ ELNQRMTQGQ SDFVFVIGGS NGLHKDVLQR |
| | Protein | SNYALSFSKM TFPHQMMRVV LIEQVYRAFK IMRGEAYHK |
| 12 | S. aureus orfX | atgaaaatca ccattttagc tgtagggaaa ctaaaagaga |
| | Accession No. | aatattggaa gcaagccata gcagaatatg aaaaacgttt |
| | AB121219 | aggcccatac accaagatag acatcataga agttccagac |
| | DNA | gaaaaagcac cagaaaatat gagcgacaaa gaaattgagc |
| | | aagtaaaaga aaaagaaggc caacgaatac tagccaaaat |
| | | caaaccacaa tccacagtca ttacattaga aatacaagga |
| | | aagatgctat cttccgaagg attggcccaa gaattgaacc |
| | | aacgcatgac ccaagggcaa agcgactttg tattcgtcat |
| | | tggcggatca aacggcctgc acaaggacgt cttacaacgc |
| | | agtaactatg cactatcatt tagcaaaatg acattcccac |
| | | atcaaatgat gcgggttgtg ttaattgaac aagtgtatag |
| | | agcatttaag attatgcgtg gagaggcgta tcataaataa |

In various embodiments, the reverse primer may comprise or consist of a sequence as set forth in any of SEQ ID NOS: 13-15 as follows or a complement thereof:

| | |
|---|---|
| SEQ ID NO: 13 | (CAAGGGCAAAGCGACTTTGT); |
| SEQ ID NO: 14 | (TGCTATCTTCCGAAGGATTGGC); |
| SEQ ID NO: 15 | (GTCATTACATTAGAAATACAAGGAAAGATGC). |

In various embodiments, the probe may be a probe that includes a sequence targeted to an orfX region adjacent to the MREJ region such as a nucleotide sequence encoding an orfX polypeptide or an orfX nucleotide sequence or a complement thereof. An example of one orfX nucleotide sequence is set forth in Table 3 above or a complement thereof. In various embodiments, the probe may comprise or consist of a sequence se set forth in

| | |
|---|---|
| SEQ ID NO: 16 | (CGGCCTGCACAAGGACGTCTTACA). |

In various embodiments, the probe may comprise or consist of a sequence of from about 10 to about 40, from about 15 to about 35 or from about 20 to about 30 contiguous nucleotides in length and, in particular, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 contiguous nucleotides in length. In various embodiments, the probe may comprise or consist of a sequence of not more than about 30 nucleotides in length and, in particular, not more than 35, not more than 34, not more than 33, not more than 32, not more than 31, not more than 30, not more than 29, not more than 28, not more than 27, not more than 26, not more than 25. In various embodiments, all of the nucleotides in the probe may be fully complementary to the target sequence of an MRSA to be detected. In various embodiments, the probe may be designed to hybridize to a conserved S. aureus orfX region. In particular, the conserved orfX region targeted by the probe may have no known single nucleotide polymoprhisms (SNPs). Such conserved regions of S. aureus orfX can be determined by comparison of S. aureus orfX sequences available in a database such as the GenBank NCBI database which is available online at the internet site http://www.ncbi.nlm.nih.gov/. Other databases may include EMBL Nucleotide Sequence Database and DNA Data Bank of Japan (DDBJ). It will be appreciated by the skilled artisan that the specific probe sequences provided herein have been designed based upon S. aureus orfX sequences available at the time of filing, however, S. aureus orfX sequences may subsequently be identified or may arise as a result of mutation such that a previously conserved region is no longer conserved. In such instances, new probe sequences may be designed to hybridize to different regions that continue to remain conserved. In various embodiments, the reverse primer may also be designed to hybridize to a conserved region.

In various embodiments, the at least one probe may include at least one linear probe such as, for example, a TAQMAN® probe. Such linear probes do not contain the stem-loop structure such as is present in molecular beacon probes.

In various embodiments, not more than one probe may be used in the method, particularly in embodiments in which the probe has been designed to hybridize to a conserved region. In other embodiments, more than one probe may be used, such as, for example where the region to which the probe hybridizes may contain one or more SNPs. In such instances, a set of probes may include a separate probe for each of the polymorphic sequences.

### Oligonucleotide Sets

The present invention also provides, in various embodiments, sets of oligonucleotides for use in a method of detecting the presence and/or amount of MRSA in a sample such as described above. The sets of oligonucleotides may include a) at least five forward primers, b) a reverse primer that is targeted to a first S. aureus orfX region and c) at least one probe of not more than 30 contiguous nucleotides in which the probe is targeted to a second S. aureus orfX region between the first S. aureus orfX region and the MREJ regions.

In various embodiments, the forward and reverse primers of the invention may independently comprise or consist of sequences of at least 10 nucleotides, at least 15 nucleotides or at least 20 nucleotides. In various embodiments, the forward and reverse primers may independently comprise or consist of sequences of from about 15 to about 40, from about 15 to about 35 or from about 20 to about 30 contiguous nucleotides in length and in particular, 15, 16, 17, 18, 19, 20, 21, 22, 23, 24, 25, 26, 27, 28, 29, 30, 31, 32, 33, 34, 35, 36, 37, 38, 39 or 40 contiguous nucleotides in length.

In various embodiments, each of the at least five forward primers may be targeted to a different one of a Type II MREJ, a Type III MREJ, a Type IV MREJ, a Type V MREJ or a Type VII MREJ such as, for example, the sequences set forth in Table 2 or complements of sequences in the table. In various embodiments, the at least five forward primers may each include a sequence of contiguous nucleotides of a different one of a Type II MREJ, a Type III MREJ, a Type IV MREJ, a Type V MREJ or a Type VII MREJ such as, for example, the sequences set forth in SEQ ID NOS: 1-5 shown in Table 2 or complements of sequences in the table. In various embodiments, the at least five forward primers may comprise or consist of sequences as set forth SEQ ID NOS: 6-10 or complements thereof.

In various embodiments, the reverse primer may include a sequence targeted to an orfX region adjacent to the MREJ region such as a nucleotide sequence encoding an orfX polypeptide or an orfX nucleotide sequence or a complement thereof. An example of one orfX nucleotide and putative polypeptide sequence is set forth in Table 3. In various embodiments, the reverse primer may comprise or consist of a sequence as set forth in any of SEQ ID NOS: 13-15 or a complement thereof.

In various embodiments, the probe may comprise or consist of a sequence of not more than about 30 nucleotides in length and, in particular, not more than 30, not more than 29, not more than 28, not more than 27, not more than 26, not more than 25. contiguous nucleotides in length and, in particular, 10, 11, 12, 13, 14, 15, 16, 17, 18, 19, 20, 21, 22, 23, 24, 25, 26, 27, 28, 29 or 30 contiguous nucleotides in length. In various embodiments, all of the nucleotides in the probe may be fully complementary to the target sequence of an MRSA to be detected. In various embodiments, the probe may be designed to hybridize to a conserved S. aureus orfX region. In particular, the conserved orfX region targeted by the probe may have no known single nucleotide polymoprhisms (SNPs).

In various embodiments, the at least one probe may include at least one linear probe such as, for example, a TAQMAN® probe. Such linear probes do not contain the stem-loop structure such as is present in molecular beacon probes.

In various embodiments, not more than one probe may be used in the method, particularly in embodiments in which the probe has been designed to hybridize to a conserved region. In other embodiments, more than one probe may be used, such as, for example where the region to which the probe hybridizes may contain one or more SNPs. In such instances, a set of probes may include a separate probe for each of the polymorphic sequences.

### Kits

In various embodiments, the present invention can also include kits. Such kits can include the compositions of the present invention and, in certain embodiments, instructions for administration. When supplied as a kit, the different components of the composition can be packaged in separate containers and admixed immediately before use. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components. In addition, if more than one route of administration is intended or more than one schedule for administration is intended, the different components can be packaged separately and not mixed prior to use. In various embodiments, the different components can be packaged in one composition for administration together.

In particular, the kits may include, in various embodiments, a lysyl endopeptidase, at least one, at least two, at least three, at least four or at least five forward primers that are targeted to an S. aureus MREJ region; a reverse primer that is targeted to a first orfX region; and at least one probe that is targeted to a second S. aureus orfX region between the first S. aureus orfX region and the MREJ region. In other embodiments, the kits may include at least five forward primers, a reverse primer that is targeted to a first orfX region and at least one probe of not more than 30 contiguous nucleotides that is targeted to a second S. aureus orfX region between the first S. aureus orfX region and the MREJ region.

In various embodiments, the forward and reverse primers of the invention may independently comprise or consist of sequences of at least 10 nucleotides, at least 15 nucleotides or at least 20 nucleotides. Further, the forward and reverse primers may independently comprise or consist of sequences of from about 15 to about 40, from about 15 to about 35 or from about 20 to about 30 contiguous nucleotides in length and in particular, 15, 16, 17, 18, 19, 20, 21, 22, 23, 24, 25, 26, 27, 28, 29, 30, 31, 32, 33, 34, 35, 36, 37, 38, 39 or 40 contiguous nucleotides in length.

In various embodiments, each of the at least one, at least two, at least three, at least four or at least five forward primers of the kits may be targeted to a different one of a Type II MREJ, a Type III MREJ, a Type IV MREJ, a Type V MREJ or a Type VII MREJ such as, for example, the sequences set forth in Table 2 or complements of sequences in the table. In various embodiments, the each of the at least one, at least two, at least three, at least four or at least five forward primers may include a sequence of contiguous nucleotides of a different one of a Type II MREJ, a Type III MREJ, a Type IV MREJ, a Type V MREJ or a Type VII MREJ such as, for example, the sequences set forth in SEQ ID NOS: 1-5 shown in Table 2 or complements of sequences in the table. In various embodiments, the primers may comprise or consist of sequences selected from SEQ ID NOS: 6-10 or complements thereof.

The reverse primer may include a sequence targeted to an orfX region adjacent to the MREJ region such as a nucleotide sequence encoding an orfX polypeptide or an orfX nucleotide sequence or a complement thereof. An example of one orfX nucleotide and putative polypeptide sequence is set forth in Table 3. In various embodiments, the reverse primer may comprise or consist of a sequence selected from SEQ ID NOS: 13-15 or a complement thereof.

The probe may comprise or consist of a sequence of not more than about 30 nucleotides in length and, in particular, not more than 30, not more than 29, not more than 28, not more than 27, not more than 26, not more than 25. contiguous nucleotides in length and, in particular, 10, 11, 12, 13, 14, 15, 16, 17, 18, 19, 20, 21, 22, 23, 24, 25, 26, 27, 28, 29 or 30 contiguous nucleotides in length. In various embodiments, all of the nucleotides in the probe may be fully complementary to the target sequence of an MRSA to be detected. In various embodiments, the probe may be designed to hybridize to a conserved S. aureus orfX region. In particular, the conserved orfX region targeted by the probe may have no known single nucleotide polymoprhisms (SNPs).

The at least one probe may include at least one linear probe such as, for example, a TAQMAN® probe. Such linear probes do not contain the stem-loop structure such as is present in molecular beacon probes.

In various embodiments, not more than one probe may be used in the method, particularly in embodiments in which the probe has been designed to hybridize to a conserved region. In other embodiments, more than one probe may be used, such as, for example where the region to which the probe hybridizes may contain one or more SNPs. In such instances, a set of probes may include a separate probe for each of the polymorphic sequences.

Kits may also include reagents in separate containers such as, for example, sterile water or saline to be added to a lyophilized active component packaged separately. For example, sealed glass ampoules may contain lyophilized phosphatases and in a separate ampoule, sterile water, sterile saline, Tris HCl-EDTA buffer (“TE buffer”) or sterile each of which has been packaged under a neutral non-reacting gas, such as nitrogen. Ampoules may consist of any suitable material, such as glass, organic polymers, such as polycarbonate, polystyrene, ceramic, metal or any other material typically employed to hold reagents. Other examples of suitable containers include bottles that may be fabricated from similar substances as ampoules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

### Biological Methods

Methods described above involving conventional molecular biology techniques are generally known in the art and are described in detail in methodology treatises such as MOLECULAR CLONING: A LABORATORY MANUAL, 2nd ed., vol. 1-3, ed. Sambrook et al., Cold Spring Harbor Laboratory Press, Cold Spring Harbor, N.Y., 1989; and CURRENT PROTOCOLS IN MOLECULAR BIOLOGY, ed. Ausubel et al., Greene Publishing and Wiley-Interscience, New York, 1992 (with periodic updates). Various techniques using polymerase chain reaction (PCR) are described, e.g., in Innis et at., PCR PROTOCOLS: A GUIDE TO METHODS AND APPLICATIONS, Academic Press San Diego, 1990. PCR-primer pairs can be derived from known sequences by known techniques such as using computer programs intended for that purpose. Methods and apparatus for chemical synthesis of nucleic acids are provided in several commercial embodiments, e.g., those provided by Applied Biosystems, Foster City, Calif., and Sigma-Genosys, The Woodlands, Tex.

### EXAMPLES

Aspects of the present teachings may be further understood in light of the following examples, which should not be construed as limiting the scope of the present teachings in any way.

### Example 1

This example illustrates an assay method of the invention for detecting MRSA in nasal swab samples.

For each test, nasal swabs containing samples from subjects were placed in 2 ml screw-cap tubes containing 1 ml of TE buffer at pH 8.0. The TE buffer was prepared from stock solutions of TRIS100× (1M: 8.17 g TRIS/50 mL double distilled water) and EDTA 100× (0.1M: 1.86 g Naₑ EDTA/50 mL double distilled water) by combining 0.5 mL TRIS100× with 45 mL double distilled water, adjusting pH to 8, adding 0.5 mL EDTA 100× and adding double distilled water up to 50 mL.

The swabs were broken after holding the swabs near the rim of the tubes and lifting them a few millimeters from the bottom. The tubes were then capped and vortexed at high speed.

The cell suspensions were then pipetted into 1.5 mL, screw-cap conical lysis tubes containing 0.1 mm glass beads. These were centrifuged followed by removal and discarding of the supernatants.

To the pellets, 54 μL of Lysis buffer containing Achromopeptidase (A3547, Sigma-Aldrich Co., St. Louis, Mo.) at a concentration of 3U/μl TE buffer were added. The lysis tubes were then vortexed at high speed for 1 minute and centrifuged briefly followed by incubated of the tubes at 37° C. for about 7 min. After vortexing again for about 3 minutes, the tubes were briefly centrifuged. The lysis tubes were then heated at 95° C. for about 5 minutes. After cooling, 15 μL aliquots of lysates were combined with 10 μL of PCR mixture were placed in 25 μL reaction tubes. The PCR mixture contained 10 μL master mix containing oligonucleotide primers and probe (Table 4) and 15 uL LightCycler-480 Probes Master (Roche Applied Science, Indianapolis, Ind.) containing FastStart Taq DNA Polymerase and four deoxyribonucleoside triphosphates. The TAQMAN® probe contained the fluorophore, 6-carboxyfluorescein (FAM) at the 5′ end and the quencher, tetramethylrhodamine, (TAMRA) at the 3′ end.

TABLE 4
Oligonucleotide Primers and Probe

| SEQ ID | | |
|---|---|---|
| NO | DESCRIPTION | SEQUENCE |
| 6 | Type II MERJ Forward Primer | AAATGAAAGACTGCGGAGGCT |
| 7 | Type III MERJ Forward Primer | ATCGTATGATATTGCAAGGTATAATCCAA |
| 8 | Type IV MERJ Forward Primer | GGAAATCCATCTCTACTTTATTGTTTTCTT |
| 9 | Type V MERJ Forward Primer | CGGCTGAAATAACCGCATCA |
| 10 | Type VII MERJ Forward Primer | TCGATTTACACACCATGTATTAAATAATGG |
| 13 | Reverse Primer | CAAGGGCAAAGCGACTTTGT |
| 16 | Probe | CGGCCTGCACAAGGACGTCTTACA |

The reaction tubes were then placed in a SmartCycler II automated real-time PCR system (Cepheid, Sunnyvale, Calif.). The thermal cycling protocol was as follows: 3 min at 95° C. for initial denaturation followed by 48 cycles of three steps consisting of 5 s at 95° C. for denaturation, 15 s at 60° C. for annealing, and 20 s at 72° C. for extension.

These above tests were performed on a large number of samples which are summarized in the following example in an analysis of the ability of the test to resolve substantially all samples tested.

### Example 2

This example illustrates use of achromopeptidase in sample preparation to substantially eliminate unresolved results in accordance with the methods of the present invention.

Test samples were assayed as described in Example 1 and the results were evaluated to assess the number of unresolved samples produced by the method. This was compared to results obtained using the commercially available BD GeneOhm MRSA assay kit (Becton, Dickinson and Company, Franklin Lakes, N.J.).

As described in Example 1, the sample processing steps prior to PCR amplification included incubation with the lysyl endopeptidase, achromopeptidase, along with vortexing the sample in the presence of microbeads and thermal treatment to lyse bacterial cells and concentrate the sample. In comparison to this, the BD GeneOhm MRSA assay method for sample processing prior to PCR amplification involved vortexing in the presence of microbeads and thermal treatment (see package insert which is available online at the internet site http://www.bd.com/geneohm/english/products/pdfs/mrsa_pkginsert.pdf). In accordance with the method described in the package insert, the samples were prepared for PCR amplification as follows. Nasal swabs containing the patient sample were placed in tubes containing buffer which were then vortexed at high speed for one minute. The cell suspensions were then transferred to a lysis tube containing microbeads and centrifuged at high speed for 5 minutes at room temperature. The supernatants were then removed and discarded. 50 μL of buffer was then added to each lysis tube which was then vortexed at high speed for 5 minutes. The lysis tubes were briefly centrifuged and then heated at 95±2° C. for two minutes. The lysis tubes were then kept on ice or on a cooling block until used in PCR amplification.

The methods described in Example 1 which included incubation with achromopeptidase, was used for one set of samples and the methods described in the BD GeneOhm package insert was used for another set of samples. The number of unresolved samples was then determined. Results are shown in Table 5.

TABLE 5
Unresolved Samples

| | Assay Method | BD GeneOhm |
|---|---|---|
| | of the Invention | MRSA Assay |
| Total No. of | 3899 | 1009 |
| Samples | | |
| No. of | 2 | 131 |
| Unresolved | | |
| Samples | | |
| % Unresolved | 0.05% | 13% |

As shown in the table, there were substantially no (0.05%) unresolved samples with method of the invention compared to 13% unresolved samples with the BD GeneOhm MRSA assay.

### REFERENCES CITED

All references cited in this specification are hereby incorporated by reference in their entireties. The discussion of the references herein is intended merely to summarize the information therein and the assertions made by their authors and no admission is made that any reference constitutes prior art relevant to patentability. Applicant reserves the right to challenge the accuracy and pertinency of the cited references.

What is claimed is:

1. A method of detecting the presence and/or amount of methicillin-resistant S. aureus (MRSA) if present in a sample, the method comprising:
a) processing the sample by contacting the sample with a lysyl endopeptidase to produce a processed sample;
b) performing an amplification reaction by contacting the processed sample with a set of primers and at least one probe to produce an amplicon and hybridizing of the at least one probe to the amplicon if the MRSA is present in the sample, the set of primers and at least one probe comprising
i) at least one forward primer that hybridizes to an S. aureus SCCmec right extremity junction (MREJ) region,
ii) a reverse primer that hybridizes to a first S. aureus orfX region, and
iii) at least one probe that hybridizes to a second S. aureus orfX region; and
c) detecting the hybridizing of the at least one probe to the amplicon as an indication of the presence and/or amount of MRSA in the sample.

2. A method according to claim 1, wherein the lysyl endopeptidase is a lysyl endopeptidase of Lysobacter sp IB-9374.

3. A method according to claim 2, wherein the lysyl endopeptidase is LepA.

4. A method according to claim 1, wherein the at least one forward primer comprises at least five forward primers comprising:
a) a forward primer that hybridizes to a type II S. aureus MREJ region,
b) a forward primer that hybridizes to a type III S. aureus MREJ region,
c) a forward primer that hybridizes to a type IV S. aureus MREJ region,
d) a forward primer that hybridizes to a type V S. aureus MREJ region, and
e) a forward primer that hybridizes to a type VII S. aureus MREJ region.

5. A method according to claim 4, wherein the at least five forward primers comprise a first sequence of at least 15 contiguous nucleotides of SEQ ID NO: 1; a second sequence of at least 15 contiguous nucleotides of SEQ ID NO: 2; a third sequence of at least 15 contiguous nucleotides of SEQ ID NO: 3; a fourth sequence of at least 15 contiguous nucleotides of SEQ ID NO: 4; and a fifth sequence of at least 15 contiguous nucleotides of SEQ ID NO: 5.

6. A method according to claim 5, wherein the at least five forward primers comprise SEQ ID NOS: 6-10.

7. A method according to claim 1, wherein the reverse primer comprises a sequence of at least 15 contiguous nucleotides of a sequence encoding SEQ ID NO: 11.

8. A method according to claim 7, wherein the reverse primer comprises a sequence of at least 15 contiguous nucleotides of SEQ ID NO: 12.

9. A method according to claim 8, wherein the reverse primer comprises SEQ ID NO: 13.

10. A method according to claim 1, wherein the at least one probe comprises a sequence of at least 15 contiguous nucleotides of a sequence encoding SEQ ID NO: 11.

11. A method according to claim 10, wherein the at least one probe comprises a sequence of at least 15 contiguous nucleotides of SEQ ID NO: 12.

12. A method according to claim 11, wherein the at least one probe comprises SEQ ID NO: 14.

13. A method of detecting the presence and/or amount of MRSA if present in a sample, the method comprising:
a) performing an amplification reaction by contacting the sample with a set of primers and at least one probe to produce an amplicon and hybridizing of the at least one probe to the amplicon if the MRSA is present in the sample, the set of primers and at least one probe comprising
i) at least five forward primers comprising
A) a forward primer that hybridizes to a type II S. aureus MREJ region,
B) a forward primer that hybridizes to a type III S. aureus MREJ region,
C) a forward primer that hybridizes to a type IV S. aureus MREJ region,
C) a forward primer that hybridizes to a type V S. aureus MREJ region, and
E) a forward primer that hybridizes to a type VII S. aureus MREJ region;
ii) a reverse primer that hybridizes to a first S. aureus orfX region; and
iii) at least one probe of not more than 30 contiguous nucleotides, wherein the probe hybridizes to a second S. aureus orfX region; and
b) detecting the hybridizing of the at least one probe to the amplicon as an indication of the presence and/or amount of MRSA in the sample.

14. A method according to claim 13, wherein the at least one probe is at least one linear probe.

15. A method according to claim 13, wherein the at least one probe hybridizes to a conserved S. aureus orfX region.

16. A method according to claim 13, wherein the at least one probe comprises not more than one probe.

17. A method according to claim 13, wherein the at least five forward primers comprise a first sequence of at least 15 contiguous nucleotides of SEQ ID NO: 1; a second sequence of at least 15 contiguous nucleotides of SEQ ID NO: 2; a third sequence of at least 15 contiguous nucleotides of SEQ ID NO: 3; a fourth sequence of at least 15 contiguous nucleotides of SEQ ID NO: 4; and a fifth sequence of at least 15 contiguous nucleotides of SEQ ID NO: 5.

18. A method according to claim 17, wherein the at least five forward primers comprise SEQ ID NOS: 6-10.

19. A method according to claim 13, wherein the reverse primer comprises a sequence of at least 15 contiguous nucleotides of a sequence encoding SEQ ID NO: 11.

20. A method according to claim 19, wherein the reverse primer comprises a sequence of at least 15 contiguous nucleotides of SEQ ID NO: 12.

21. A method according to claim 20, wherein the reverse primer comprises SEQ ID NO: 13.

22. A method according to claim 13, wherein the at least one probe comprises a sequence of at least 15 contiguous nucleotides of a sequence encoding SEQ ID NO: 11.

23. A method according to claim 22, wherein the at least one probe comprises a sequence of at least 15 contiguous nucleotides of SEQ ID NO: 12.

24. A method according to claim 23, wherein the at least one probe comprises SEQ ID NO: 14.

25. A method of detecting the presence and/or amount of methicillin-resistant S. aureus (MRSA) if present in a sample, the method comprising:
a) processing the sample by contacting the sample with a lysyl endopeptidase to produce a processed sample;
b) performing an amplification reaction by contacting the processed sample with a set of primers that amplify a target sequence comprising an S. aureus SCCmec right extremity junction (MREJ) region and an adjacent orfX region of an MRSA to produce an amplicon, wherein the set of primers comprise:
i) at least one forward primer that targets an S. aureus MREJ region, and
ii) a reverse primer that targets an S. aureus orfX region; and
c) detecting the amplicon with at least one probe that hybridizes to the amplified orfX region of the amplicon as an indication of the presence and/or amount of MRSA in the sample.

26. A method according to claim 25, wherein the lysyl endopeptidase is a lysyl endopeptidase of Lysobacter sp IB-9374.

27. A method according to claim 25, wherein the lysyl endopeptidase is LepA.

28. A method according to claim 25, wherein the at least one forward primer comprises at least five forward primers comprising:
a) a forward primer that targets a type II S. aureus MREJ region,
b) a forward primer that targets a type III S. aureus MREJ region,
c) a forward primer that targets a type IV S. aureus MREJ region,
d) a forward primer that targets a type V S. aureus MREJ region, and
e) a forward primer that targets a type VII S. aureus MREJ region; and

29. A method according to claim 28, wherein the at least five forward primers comprise a first sequence of at least 15 contiguous nucleotides of SEQ ID NO: 1; a second sequence of at least 15 contiguous nucleotides of SEQ ID NO: 2; a third sequence of at least 15 contiguous nucleotides of SEQ ID NO: 3; a fourth sequence of at least 15 contiguous nucleotides of SEQ ID NO: 4; and a fifth sequence of at least 15 contiguous nucleotides of SEQ ID NO: 5.

30. A method according to claim 29, wherein the at least five forward primers comprise SEQ ID NOS: 6-10.

31. A method according to claim 25, wherein the reverse primer comprises a sequence of at least 15 contiguous nucleotides of a sequence encoding SEQ ID NO: 11.

32. A method according to claim 31, wherein the reverse primer comprises a sequence of at least 15 contiguous nucleotides of SEQ ID NO: 12.

33. A method according to claim 32, wherein the reverse primer comprises SEQ ID NO: 13.

34. A method according to claim 25, wherein the at least one probe comprises a sequence of at least 15 contiguous nucleotides of a sequence encoding SEQ ID NO: 11.

35. A method according to claim 34, wherein the at least one probe comprises a sequence of at least 15 contiguous nucleotides of SEQ ID NO: 12.

36. A method according to claim 35, wherein the at least one probe comprises a sequence of at least 20 contiguous nucleotides of SEQ ID NO: 12.

37. A method of detecting the presence and/or amount of methicillin-resistant S. aureus (MRSA) if present in a sample, the method comprising:
a) performing an amplification reaction by contacting the sample with a set of primers that amplify a target sequence comprising an S. aureus SCCmec right extremity junction (MREJ) region and an adjacent orfX region of an MRSA to produce an amplicon, the set of primers comprising
i) at least five forward primers comprising
A) a forward primer that targets a type II S. aureus MREJ region,
B) a forward primer that targets a type III S. aureus MREJ region,
C) a forward primer that targets a type IV S. aureus MREJ region,
D) a forward primer that targets a type V S. aureus MREJ region, and
E) a forward primer that targets a type VII S. aureus MREJ region; and
ii) a reverse primer that targets an S. aureus orfX region; and
c) detecting the amplicon with at least one probe of not more than 30 contiguous nucleotides that hybridize to the amplified orfX region as an indication of the presence and/or amount of MRSA in the sample.

38. A method according to claim 37, wherein the at least one probe is at least one linear probe.

39. A method according to claim 37, wherein the at least one probe hybridizes to a conserved region of the amplified orfX region.

40. A method according to claim 37, wherein the at least one probe comprises not more than one probe.

41. A method according to claim 37, wherein the at least five forward primers comprise a first sequence of at least 15 contiguous nucleotides of SEQ ID NO: 1; a second sequence of at least 15 contiguous nucleotides of SEQ ID NO: 2; a third sequence of at least 15 contiguous nucleotides of SEQ ID NO: 3; a fourth sequence of at least 15 contiguous nucleotides of SEQ ID NO: 4; and a fifth sequence of at least 15 contiguous nucleotides of SEQ ID NO: 5.

42. A method according to claim 41, wherein the at least five forward primers comprise SEQ ID NOS: 6-10.

43. A method according to claim 37, wherein the reverse primer comprises a sequence of at least 15 contiguous nucleotides of a sequence encoding SEQ ID NO: 11.

44. A method according to claim 43, wherein the reverse primer comprises a sequence of at least 15 contiguous nucleotides of SEQ ID NO: 12.

45. A method according to claim 44, wherein the reverse primer comprises SEQ ID NO: 13.

46. A method according to claim 37, wherein the at least one probe comprises a sequence of at least 15 contiguous nucleotides of a sequence encoding SEQ ID NO: 11.

47. A method according to claim 46, wherein the at least one probe comprises a sequence of at least 15 contiguous nucleotides of SEQ ID NO: 12.

48. A method according to claim 47, wherein the at least one probe comprises SEQ ID NO: 14.

49. A set of oligonucleotides comprising:
i) at least five forward primers comprising
a) a forward primer that hybridizes to a type II S. aureus MREJ region,
b) a forward primer that hybridizes to a type III S. aureus MREJ region,
c) a forward primer that hybridizes to a type IV S. aureus MREJ region,
d) a forward primer that hybridizes to a type V S. aureus MREJ region, and
e) a forward primer that hybridizes to a type VII S. aureus MREJ region;
ii) a reverse primer that hybridizes to a first S. aureus orfX region; and
iii) at least one probe of not more than 30 contiguous nucleotides, wherein the probe hybridizes to a second S. aureus orfX region between the first S. aureus orfX region and the MREJ regions.

50. A set of oligonucleotides according to claim 49, wherein the at least one probe is at least one linear probe.

51. A set of oligonucleotides according to claim 49, wherein the at least one probe hybridizes to a conserved S. aureus orfX region.

52. A set of oligonucleotides according to claim 49 comprising not more than one probe.

53. A set of oligonucleotides according to claim 49, wherein the at least five forward primers comprise a first sequence of at least 15 contiguous nucleotides of SEQ ID NO: 1; a second sequence of at least 15 contiguous nucleotides of SEQ ID NO: 2; a third sequence of at least 15 contiguous nucleotides of SEQ ID NO: 3; a fourth sequence of at least 15 contiguous nucleotides of SEQ ID NO: 4; and a fifth sequence of at least 15 contiguous nucleotides of SEQ ID NO: 5.

54. A set of oligonucleotides according to claim 53, wherein the at least five forward primers comprise SEQ ID NOS: 6-10.

55. A set of oligonucleotides according to claim 49, wherein the reverse primer comprises a sequence of at least 15 contiguous nucleotides of a sequence encoding SEQ ID NO: 11.

56. A set of oligonucleotides according to claim 55, wherein the reverse primer comprises a sequence of at least 15 contiguous nucleotides of SEQ ID NO: 12.

57. A set of oligonucleotides according to claim 56, wherein the reverse primer comprises SEQ ID NO: 13.

58. A set of oligonucleotides according to claim 49, wherein the at least one probe comprises a sequence of at least 15 contiguous nucleotides of a sequence encoding SEQ ID NO: 11.

59. A set of oligonucleotides according to claim 58, wherein the at least one probe comprises a sequence of at least 15 contiguous nucleotides of SEQ ID NO: 12.

60. A set of oligonucleotides according to claim 59, wherein the at least one probe comprises SEQ ID NO: 14.

61. A kit for detecting the presence and/or amount of MRSA if present in a sample, the kit comprising:
a) a lysyl endopeptidase;
b) at least one forward primer that hybridizes to an S. aureus MREJ region;
c) a reverse primer that hybridizes to a first orfX region; and
d) at least one probe that hybridizes to a second S. aureus orfX region between the first S. aureus orfX region and the MREJ region.

62. A kit according to claim 61, further comprising reagents suitable for performing a PCR reaction that produces an amplicon and hybridizing of the at least one probe to the amplicon if the MRSA is present in the sample.

63. A kit according to claim 61, wherein the lysyl endopeptidase is a lysyl endopeptidase of Lysobacter sp IB-9374.

64. A kit according to claim 61, wherein the lysyl endopeptidase is LepA.

65. A kit according to claim 61 wherein the at least one forward primer comprises at least five forward primers comprising
i) a forward primer that hybridizes to a type II S. aureus MREJ region,
ii) a forward primer that hybridizes to a type III S. aureus MREJ region,
iii) a forward primer that hybridizes to a type IV S. aureus MREJ region,
iv) a forward primer that hybridizes to a type V S. aureus MREJ region, and
v) a forward primer that hybridizes to a type VII S. aureus MREJ region.

66. A kit according to claim 65, wherein the at least five forward primers comprise a first sequence of at least 15 contiguous nucleotides of SEQ ID NO: 1; a second sequence of at least 15 contiguous nucleotides of SEQ ID NO: 2; a third sequence of at least 15 contiguous nucleotides of SEQ ID NO: 3; a fourth sequence of at least 15 contiguous nucleotides of SEQ ID NO: 4; and a fifth sequence of at least 15 contiguous nucleotides of SEQ ID NO: 5.

67. A kit according to claim 66, wherein the at least five forward primers comprise SEQ ID NOS: 6-10.

68. A kit according to claim 61, wherein the reverse primer comprises a sequence of at least 15 contiguous nucleotides of a sequence encoding SEQ ID NO: 11.

69. A kit according to claim 68, wherein the reverse primer comprises a sequence of at least 15 contiguous nucleotides of SEQ ID NO: 12.

70. A kit according to claim 69, wherein the reverse primer comprises SEQ ID NO: 13.

71. A kit according to claim 61, wherein the at least one probe comprises a sequence of at least 15 contiguous nucleotides of a sequence encoding SEQ ID NO: 11.

72. A kit according to claim 71, wherein the at least one probe comprises a sequence of at least 15 contiguous nucleotides of SEQ ID NO: 12.

73. A kit according to claim 72, wherein the at least one probe comprises SEQ ID NO: 14.

74. A kit for detecting the presence and/or amount of MRSA if present in a sample, the kit comprising:
a) at least five forward primers comprising
i) a forward primer that hybridizes to a type II S. aureus MREJ region,
ii) a forward primer that hybridizes to a type III S. aureus MREJ region,
iii) a forward primer that hybridizes to a type IV S. aureus MREJ region,
iv) a forward primer that hybridizes to a type V S. aureus MREJ region, and
v) a forward primer that hybridizes to a type VII S. aureus MREJ region;
b) a reverse primer that hybridizes to a first orfX region; and
c) at least one probe of not more than 30 contiguous nucleotides that hybridize to a second S. aureus orfX region between the first S. aureus orfX region and the MREJ region.

75. A kit according to claim 74, further comprising reagents suitable for performing a PCR reaction that produces an amplicon and hybridizing of the at least one probe to the amplicon if the MRSA is present in the sample.

76. A kit according to claim 74, wherein the at least one probe is at least one linear probe.

77. A kit according to claim 74, wherein the at least one probe hybridizes to a conserved S. aureus orfX region.

78. A kit according to claim 74, wherein the at least one probe comprises not more than one probe.

79. A kit according to claim 74, wherein the at least five forward primers comprise a first sequence of at least 15 contiguous nucleotides of SEQ ID NO: 1; a second sequence of at least 15 contiguous nucleotides of SEQ ID NO: 2; a third sequence of at least 15 contiguous nucleotides of SEQ ID NO: 3; a fourth sequence of at least 15 contiguous nucleotides of SEQ ID NO: 4; and a fifth sequence of at least 15 contiguous nucleotides of SEQ ID NO: 5.

80. A kit according to claim 79, wherein the at least five forward primers comprise SEQ ID NOS: 6-10.

81. A kit according to claim 74, wherein the reverse primer comprises a sequence of at least 15 contiguous nucleotides of a sequence encoding SEQ ID NO: 11.

82. A kit according to claim 81, wherein the reverse primer comprises a sequence of at least 15 contiguous nucleotides of SEQ ID NO: 12.

83. A kit according to claim 82, wherein the reverse primer comprises SEQ ID NO: 13.

84. A kit according to claim 74, wherein the at least one probe comprises a sequence of at least 15 contiguous nucleotides of a sequence encoding SEQ ID NO: 11.

85. A kit according to claim 84, wherein the at least one probe comprises a sequence of at least 15 contiguous nucleotides of SEQ ID NO: 12.

86. A kit according to claim 85, wherein the at least one probe comprises SEQ ID NO: 14.
